(19) United States
(12) Patent Application Publication
(10) Pub. No.: US 2025/0213632 A1
(43) Pub. Date: Jul. 3, 2025

(54) COMPOSITION FOR AMELIORATING SIDE EFFECTS CAUSED BY CANCER TREATMENT COMPRISING LACTOBACILLUS FERMENTUM HEM1036 STRAIN AS ACTIVE INGREDIENT

(71) Applicant: HEM PHARMA INC., Pohang-si (KR)

(72) Inventors: Yo Sep JI, Suwon-si (KR); So Young PARK, Suwon-si (KR); Hye Ji KANG, Suwon-si (KR); Bo Bae KIM, Suwon-si (KR)

(21) Appl. No.: 19/087,756

(22) Filed: Mar. 24, 2025

Related U.S. Application Data
(63) Continuation of application No. PCT/KR2023/015076, filed on Sep. 27, 2023.

(30) Foreign Application Priority Data
Sep. 30, 2022 (KR) 10-2022-0124926

(51) Int. Cl. A61K 35/747; A61K 35/00; A61P 1/12; A61P 3/02; C12N 1/20; C12R 1/225

(52) U.S. Cl. CPC A61K 35/747; A61P 1/12; A61P 3/02; C12N 1/205; A61K 2035/115; C12R 2001/225

(57) ABSTRACT

A composition for ameliorating side effects of cancer treatment, comprising a strain Lactobacillus fermentum HEM1036 (KCTC13978BP) as an active ingredient according to an embodiment of the present disclosure can prevent or treat side effects caused by cancer treatment or intake of anti-cancer agents through regulation of short-chain fatty acids and modulation of gut microbiota, and the composition can be applied to food compositions, health functional food compositions, pharmaceutical compositions, etc. Under the provisions of the Budapest Treaty on the International Recognition of the Deposit of Microorganisms for the Purposes of Patent Procedure, the Lactobacillus fermentum HEM1036 strain was deposited with the international depositary authority: the Korean Collection for Type Cultures on Oct. 8, 2019, under the Accession Number: KCTC 13978BP.

### TECHNICAL FIELD

The present disclosure relates to a composition for ameliorating side effects of cancer treatment, comprising a strain Lactobacillus fermentum HEM1036 as an active ingredient. In particular, the present disclosure relates to a composition for ameliorating low anterior resection syndrome (LARS), diarrhea, or weight loss.

### BACKGROUND

According to the recently published national cancer statistics, the incidence rate of colon cancer in 2019 was 56.5 cases per 100,000 people, ranking fourth after thyroid cancer (59.8), lung cancer (58.4), and stomach cancer (57.4) (crude incidence rate). In 2019 alone, 29,030 people were newly diagnosed with colorectal cancer. Colorectal cancer risk factors, such as red meat and alcohol, can lead to the formation of polyps in the colonic mucosa, some of which may develop into colorectal cancer. The incidence also tends to increase with age, and most cases occur in individuals over 50 years old.

During surgery for rectal cancer, which is a type of colorectal cancer, tissue at least 10 cm above and about 2 cm below the tumor is resected. Thus, a part of the rectum and sigmoid colon is removed depending on its location, and the remaining sigmoid colon or descending colon is pulled down and connected to the remaining rectum or anus. This surgery, which allows feces to pass through the anus, is referred to as “low anterior resection”. Various defecation disorders that occur after the surgery are collectively referred to as “low anterior resection syndrome (LARS)”. Symptoms of LARS are most severe immediately after surgery and gradually ameliorated over 1 to 2 years, but they can never fully recover to normal. Therefore, defecation dysfunction after rectal cancer surgery is considered a permanent sequela.

The exact cause of LARS has not yet been clearly identified, but it seems to result from a combination of factors. First, a reduced rectum length leads to a decrease in fecal storage capacity. In a normal person, feces accumulates in the rectum and is expelled all at once. However, in a rectal cancer surgery patient with a shortened residual rectum, the fecal storage capacity is reduced, which leads to frequent, small-volume bowel movements. Second, during rectal cancer surgery, some nerves involved in the movement of the rectum, sigmoid colon, and descending colon are inevitably severed, which results in impaired motility of the remaining rectum and the sigmoid colon and descending colon reconnected thereto. Third, some rectal cancer patients experience reduced anal sphincter function after surgery, which results in a decrease in ability to contract the anus. This also seems to be the cause of defecation disorders, such as fecal incontinence.

Meanwhile, diarrhea is one of the most common side effects experienced by cancer patients undergoing pharmacological therapy. Diarrhea is water feces that comes three or four times in one day and accompanied by abdominal pain, perineal discomfort, and fecal incontinence, and can lead to electrolyte imbalance, dehydration, and kidney dysfunction and pose serious life-threatening risks. In general, chemotherapy agents slow or halt the growth of cancer cells. However, since they are not specific to cancer, they also affect normal cells and cause side effects. Targeted agents have similar side effects. These agents are classified into two major types: tyrosine kinase inhibitors (TKIs), which block important cell signaling proteins involved in various biological activities, such as cell proliferation and migration; and monoclonal antibodies, which block receptor binding on the cell surface. Although targeted agents were initially developed with a slogan of “Selectively attacking cancer cells” and expected to have lower toxicity, the commercialization of these agents has revealed significant toxicity and, in some cases, fatal side effects.

Anti-cancer agent-induced DNA damage in intestinal mucosal cells triggers cell death, and reactive oxygen species (ROS) and inflammatory factors accumulate in tissue and lead to tissue damage, inflammation, and ulcer. Also, the damaged intestinal barrier creates an environment where bacteria can easily permeate, which exacerbates inflammation and leads to mucositis and diarrhea.

In addition to LARS and diarrhea, many other side effects occur due to cancer treatments or intake of anti-cancer agents. In particular, severe weight loss is common among cancer patients.

In this regard, Korean Patent No. 10-2215592 proposed a method for improving gut environment using Lactobacillus fermentum HEM1036, but did not specifically address the effects on LARS, diarrhea, or weight loss.

### DISCLOSURE OF THE INVENTION

### Problems to be Solved by the Invention

The present disclosure is conceived to provide a composition for ameliorating side effects of cancer treatment. In particular, the present disclosure provides the composition to ameliorate LARS, diarrhea, or weight loss.

### Means for Solving the Problems

A first aspect of the present disclosure provides a composition for ameliorating side effects of cancer treatment, comprising a strain Lactobacillus fermentum HEM1036 (KCTC13978BP) as an active ingredient.

A second aspect of the present disclosure provides a food composition, a health functional food composition and a pharmaceutical composition for ameliorating side effects of cancer treatment, comprising a strain Lactobacillus fermentum HEM1036 (KCTC13978BP) as an active ingredient.

A third aspect of the present disclosure provides a method for ameliorating side effects of cancer treatment, comprising administering the composition.

In each aspect, the side effects of cancer treatment refer to LARS, diarrhea, or weight loss.

### Effects of the Invention

A composition for ameliorating side effects of cancer treatment, comprising a strain Lactobacillus fermentum HEM1036 (KCTC13978BP) as an active ingredient according to an embodiment of the present disclosure can prevent or treat side effects caused by cancer treatment or intake of anti-cancer agents through regulation of short-chain fatty acids and modulation of gut microbiota, and the composition can be applied to food compositions, health functional food compositions, pharmaceutical compositions, etc.

### BRIEF DESCRIPTION OF THE DRAWINGS

FIG. 1A shows the result of comparing changes in fecal short-chain fatty acids before and after administration of HEM1036.

FIG. 1B shows the correlation between fecal short-chain fatty acids and LARS scores before and after administration of HEM1036.

FIG. 2 shows the result of analyzing fecal short-chain fatty acids in normal people and LARS patents before administration of HEM1036.

FIG. 3 shows the result of analyzing short-chain fatty acid production capacity in normal people and LARS patents after administration of HEM1036.

FIG. 4A shows a questionnaire for measuring LARS scores.

FIG. 4B shows the result of comparing LARS scores before and after administration of HEM1036.

FIG. 5 shows the result of comparing quality of life scores in cancer patients before and after administration of HEM1036.

FIG. 6 shows the result of comparing changes in gut microbiota richness before and after administration of HEM1036.

FIG. 7A shows the result of a test on improving anti-cancer agent-induced weight loss after treatment with Lactobacillus fermentum HEM1036.

FIG. 7B shows the result of a test on ameliorating anti-cancer agent-induced diarrhea after treatment with Lactobacillus fermentum HEM1036.

FIG. 7C shows the result of a test on inhibiting anti-cancer agent-induced intestinal length reduction after treatment with Lactobacillus fermentum HEM1036.

FIG. 7D shows the result of ameliorating anti-cancer agent-induced diarrhea after treatment with Lactobacillus fermentum HEM1036.

FIG. 7E shows the result of observing anti-cancer agent-induced inflammatory tissues after treatment with Lactobacillus fermentum HEM1036.

FIG. 8A shows the result of checking the expression levels of a tight junction protein (occludin) of the colon in anti-cancer agent-induced diarrhea models through quantitative real-time PCR (qRT-PCR).

FIG. 8B shows the result of checking the expression levels of a tight junction protein (ZO-1) of the colon in anti-cancer agent-induced diarrhea models through qRT-PCR.

FIG. 8C shows the result of checking the expression levels of the tight junction protein (occludin) of the colon in anti-cancer agent-induced diarrhea models through immunohistochemistry.

FIG. 8D shows the result of checking the expression levels of an inflammatory cytokine (TNF-α) of the colon in anti-cancer agent-induced diarrhea models through qRT-PCR.

FIG. 8E shows the result of checking the expression levels of an inflammatory cytokine (IL-1β) in anti-cancer agent-induced diarrhea models through qRT-PCR.

FIG. 9A shows the result of a test on checking the recovery of small intestinal villi length in anti-cancer agent-induced diarrhea models.

FIG. 9B shows the result of a test on checking the recovery of small intestinal villi length (villi/crypt ratio) in anti-cancer agent-induced diarrhea models.

FIG. 9C shows the result of a test on checking the recovery of small intestinal villi structure in anti-cancer agent-induced diarrhea models.

FIG. 10A shows the result of checking the expression levels of an electrolyte balance-related gene (Clca1) of the colon in anti-cancer agent-induced diarrhea models through qRT-PCR.

FIG. 10B shows the result of checking the expression levels of an electrolyte balance-related gene (CFTR) of the colon in anti-cancer agent-induced diarrhea models through qRT-PCR.

FIG. 10C shows the result of checking the expression levels of an electrolyte balance-related gene (ANO1) of the colon in anti-cancer agent-induced diarrhea models through qRT-PCR.

FIG. 10D shows the result of checking the expression levels of an electrolyte balance-related gene (NKCC1) of the colon in anti-cancer agent-induced diarrhea models through qRT-PCR.

FIG. 10E shows the result of checking the expression levels of an electrolyte balance-related gene (NHE3) of the colon in anti-cancer agent-induced diarrhea models through qRT-PCR.

FIG. 10F shows the result of checking the expression levels of an electrolyte balance-related gene (SGLT1) of the colon in anti-cancer agent-induced diarrhea models through qRT-PCR.

FIG. 10G shows the result of checking the expression levels of an electrolyte balance-related gene (DRA) of the colon in anti-cancer agent-induced diarrhea models through qRT-PCR.

FIG. 11A shows the result of checking a decrease in cancer burden after treatment with an anti-cancer agent (5FU) and L. fermentum HEM1036 in cancer chemotherapy models.

FIG. 11B shows the result of checking the inhibition of anti-cancer agent-induced weight loss after treatment with L. fermentum HEM1036 in cancer chemotherapy models.

FIG. 11C shows the result of checking the improvement of anti-cancer agent-induced weight loss after treatment with L. fermentum HEM1036 in cancer chemotherapy models.

FIG. 11D shows the result of checking the improvement of anti-cancer agent-induced off-tumor weight loss after treatment with L. fermentum HEM1036 in cancer chemotherapy models.

FIG. 11E shows the result of checking changes in tumor weight after treatment with L. fermentum HEM1036 in cancer chemotherapy models.

### BEST MODE FOR CARRYING OUT THE INVENTION

A first aspect of the present disclosure provides a composition for ameliorating side effects of cancer treatment, comprising a strain Lactobacillus fermentum HEM1036 (KCTC13978BP) as an active ingredient.

A second aspect of the present disclosure provides a food composition, a health functional food composition and a pharmaceutical composition for ameliorating side effects of cancer treatment, comprising a strain Lactobacillus fermentum HEM1036 (KCTC13978BP) as an active ingredient.

A third aspect of the present disclosure provides a method for ameliorating side effects of cancer treatment, comprising administering the composition.

The features described above in respect of the first aspect may equally apply to the compositions according to other aspects.

### MODE FOR CARRYING OUT THE INVENTION

Hereafter, examples will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Through the whole document, the term “on” that is used to designate a position of one component with respect to another component includes both a case that the one component is adjacent to the other component and a case that any other component exists between these two components.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereafter, embodiments and examples of the present disclosure will be described in detail with reference to the accompanying drawings. However, the present disclosure may not be limited to the following embodiments, examples, and drawings.

The present disclosure relates to the use of HEM1036 for ameliorating side effects of cancer treatment, particularly LARS, diarrhea, or weight loss, disclosed in Korean Patent No. 10-2215592. According to Korean Patent No. 10-2215592, HEM1036 is used to improve the gut environment and can increase beneficial short-chain fatty acids, such as butyrate and propionate, and decrease harmful short-chain fatty acids, such as isobutyrate.

However, Korean Patent No. 10-2215592 focuses on improving the gut environment in normal people, and the disclosed strain can improve the gut environment. However, Korean Patent No. 10-2215592 does not specifically disclose how the disclosed strain ameliorates LARS or diarrhea in colorectal cancer patients. Further, it does not address the effect of ameliorating weight loss.

The present applicant analyzed fecal short-chain fatty acids of actual LARS patients and found a significantly higher level compared to the normal group (Example 2). Based on this, the present applicant conducted an HEM1036 intake test on colorectal cancer patents, particularly those who had undergone low anterior resection (LAR), to confirm that unlike the disclosure of Korean Patent No. 10-2215592, HEM1036 does not simply increase beneficial short-chain fatty acids and decrease harmful ones, but decreases the levels of total short-chain fatty acid (total SCFA) and acetate to normal levels (Example 3).

Besides, it was confirmed that the composition according to the present disclosure also ameliorates other symptoms, such as intestinal permeability inflammation and reduction in small intestinal villi length, related to LARS, diarrhea, or weight loss.

A first aspect of the present disclosure provides a pharmaceutical composition for ameliorating side effects of cancer treatment, comprising a strain Lactobacillus fermentum HEM1036 (KCTC13978BP) as an active ingredient.

A second aspect of the present disclosure provides a food composition, a health functional food composition and a pharmaceutical composition for ameliorating side effects of cancer treatment, comprising a strain Lactobacillus fermentum HEM1036 (KCTC13978BP) as an active ingredient. The features described above in respect of the first aspect may equally apply to the compositions according to another aspect.

In each aspect, the side effects of cancer treatment refer to low anterior resection syndrome (LARS), diarrhea, or weight loss.

In an embodiment of the present disclosure, the composition may decrease the levels of total SCFA and acetate.

In an embodiment of the present disclosure, the composition may decrease the expression level of TNFα or IL-1β.

In an embodiment of the present disclosure, the composition may increase the expression of one or more selected from the protein group consisting of Clca1, CFTR, ANO1, NKCC1, NHE3, SGLT1, and DRA.

Through the whole document, the term “treat” refers to all activities reducing or alleviating side effects induced by cancer treatment or intake of anti-cancer agent by administering a pharmaceutical composition for ameliorating side effects of cancer treatment, which comprises a strain Lactobacillus fermentum HEM1036 (KCTC13978BP) of the present disclosure as an active ingredient, to a subject who underwent cancer treatment or took an antibiotic.

Through the whole document, the term “low anterior resection syndrome (LARS)” refers to bowel habit changes or dyschezia that occurs after anterior resection, low anterior resection or ultra-low anterior resection which is a surgery for sigmoid colon and rectal cancer.

In an embodiment of the present disclosure, the pharmaceutical composition may be formulated and used in the form of oral dosage forms, such as powders, granules, tablets, capsules, suspensions, emulsions, syrups, aerosols, etc., external preparations, suppositories, or sterile injection solutions, according to a conventional method, but may not be limited thereto.

In an embodiment of the present disclosure, the pharmaceutical composition may be formulated with generally used diluents or excipients, such as fillers, bulking agents, binders, wetting agents, disintegrating agents or surfactants, but may not be limited thereto.

In an embodiment of the present disclosure, solid formulations for oral administration may include tablets, pills, powders, granules or capsules, and these solid formulations may be prepared by mixing heat-killed bacteria of the strain with at least one of excipients, such as starch, calcium carbonate, sucrose, lactose or gelatin. Except for the simple excipients, lubricants, such as magnesium stearate or talc, may be used, but the present disclosure may not be limited thereto.

In an embodiment of the present disclosure, liquid formulations for oral administration may include suspensions, solutions for internal use, emulsions and syrups, and may contain various excipients, such as wetting agents, sweeteners, aromatics and preservatives, in addition to generally used simple diluents, such as water and liquid paraffin, but may not be limited thereto.

In an embodiment of the present disclosure, formulations for parenteral administration may include sterilized aqueous solutions, water-insoluble excipients, suspensions, emulsions, lyophilized preparations and suppositories, but may not be limited thereto. For example, the water insoluble excipients or suspensions may contain propylene glycol, polyethylene glycol, vegetable oil, such as olive oil, injectable ester, such as ethylolate, and the like, but may not be limited thereto. For example, the suppositories may contain witepsol, macrogol, tween 61, cacao butter, laurin butter, glycerol, gelatin, and the like, but may not be limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to the Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but may not be limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with an ingredient known for treating side effects of cancer treatment. It is important to administer an amount to obtain a maximum effect in a minimum amount without side effects by considering all of the above-described factors.

In an embodiment of the present disclosure, an administration dose of the pharmaceutical composition may be determined by a person with ordinary skill in the art in view of purpose of use, severity of the disease, a patient's age, body weight, gender, medical history or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

In an embodiment of the present disclosure, the composition may contain a strain Lactobacillus fermentum HEM1036, live cells, heat-killed cells, cultured product, fragments and/or extracts thereof.

Through the whole document, the term “heat-killed bacteria” is opposite to the term “live bacteria” and refers to bodies obtained by suppressing the growth of bacteria, such as heat-treating live bacteria obtained by fermentation and metabolites thereof. The heat-killed bacteria may contain cytoplasm, cell wall, antibacterial substances, such as bacteriocin, polysaccharides, organic acid, and the like. Products using the heat-killed bacteria have higher stability than live bacteria products and are particularly excellent in heat resistance and have high stability to the external environment. Therefore, the products using the heat-killed bacteria are easier to store and have longer shelf life than the existing live bacteria products. Further, since the regulations on the use of antibiotics become stricter, there are a handful of companies that have produced heat-killed bacteria products. Therefore, considering the application as substitutes and the number of the producing companies, the marketability and growth potential is very high.

Through the whole document, the term “cultured product” refers to a substance obtained by culturing the strain of the present disclosure in a known liquid medium or solid medium and may be interchangeably used with “culture fluid”.

Through the whole document, the term “food” may include meats, sausages, breads, chocolates, candies, snacks, cookies, pizza, ramens, other noodles, gums, dairy products including ice cream, soups, beverages, teas, drinks, alcohol drinks, vitamin complexes, health functional foods and health foods, and may include all foods in the accepted meaning.

Through the whole document, the term “health functional food” refers to foods prepared and processed using raw materials or ingredients having useful functions to the human body in accordance with the Health Functional Food Act, No. 6727, and the “functionality” refers to adjusting nutrients on a structure and a function of the human body or obtaining a useful effect for health, such as a physiological action.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared in a variety of formulations. Since the food is used as raw materials, unlike general drugs, the food composition is free from side effects which may occur when a drug is taken for a long time, and may have excellent portability. Therefore, the food of the present disclosure may be taken as a supplement for enhancing the effects of improving the gut environment.

The health food refers to a food having effects of actively maintaining or promoting health conditions, as compared with general foods, and a health supplement food refers to a food for supplementing health. If necessary, the health functional food, health food and health supplement food may be interchangeably used with each other. Specifically, the health functional food is a food prepared by adding Lactobacillus fermentum HEM1036 of the present disclosure to food materials, such as beverages, teas, spices, gums, confectionery, etc., or prepared in a capsule, a powder or a suspension form. The health functional food means that it has a specific effect on health when consumed, but unlike general drugs, the health functional food is free from side effects that may occur when a drug is taken for a long time since the food is used as raw materials.

Since the food composition of the present disclosure can be routinely ingested, the food composition is expected to show a high efficacy on the improvement of depression and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions to improve smell, taste, visuality, etc. For example, the food composition may contain vitamins A, C, D, E, B1, B2, B6, B12, niacin, biotin, folate, pantothenic acid, etc. Furthermore, the food composition may also contain minerals, such as zinc (Zn), iron (Fe), calcium (Ca), chromium (Cr), magnesium (Mg), manganese (Mn), copper (Cu), chromium (Cr), etc. Moreover, the food composition may also contain amino acids, such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

The strain Lactobacillus fermentum HEM1036 of the present disclosure may be added as it is, or may be used in conjunction with other foods or food ingredients, and may be appropriately used according to a conventional method. The mixing amount of active ingredients may be appropriately determined depending on the purpose of use (prophylactic, health or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides, such as glucose and fructose; disaccharides, such as maltose and sucrose; polysaccharides, such as dextrin and cyclodextrin; and sugar alcohols, such as xylitol, sorbitol and erythritol. The sweeteners may be natural sweetener, such as thaumatin or a stevia extract; or synthetic sweetener, such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols or carbonating agents. Moreover, the health beverage composition may contain fruit flesh used to prepare natural fruit juices, fruit juice beverages or vegetable beverages. These ingredients may be used individually or in combination. A proportion of the additives is not critical, but is generally selected from 0.01 part by weight to 0.1 part by weight per 100 parts by weight of the health beverage composition of the present disclosure.

The food composition of the present disclosure may contain Lactobacillus fermentum HEM1036 of the present disclosure in a variety of % by weight as long as it can exhibit the effect of preventing hair loss and promoting hair growth. Specifically, Lactobacillus fermentum HEM1036 of the present disclosure may be contained in an amount of 0.00001% by weight to 100% by weight or 0.01% by weight to 80% by weight based on the total weight of the food composition, but may not be limited thereto.

A third aspect of the present disclosure provides a method for ameliorating side effects of cancer treatment, comprising administering the composition to a subject in need.

Through the whole document, the term “subject” may have undergone cancer treatment or taken an antibiotic.

Hereinafter, the present disclosure will be explained in more detail with reference to Examples. However, the following Examples are illustrative only for better understanding of the present disclosure, but do not limit the present disclosure.

### EXAMPLES

### Example 1. Comparison of Changes in Short-Chain Fatty Acid (SCFA) Levels Before and After Intake of Strain by Study Subject Group

### (1) Analysis of SCFA

The study compared the total SCFA, specifically acetate, propionate, and butyrate, in feces before and after intake of the composition of the present disclosure by a study subject group (a group of colorectal cancer patients who had undergone LAR and completed chemotherapy).

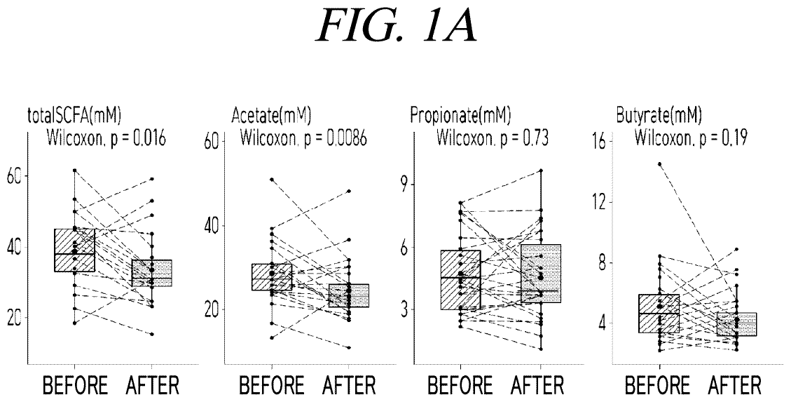

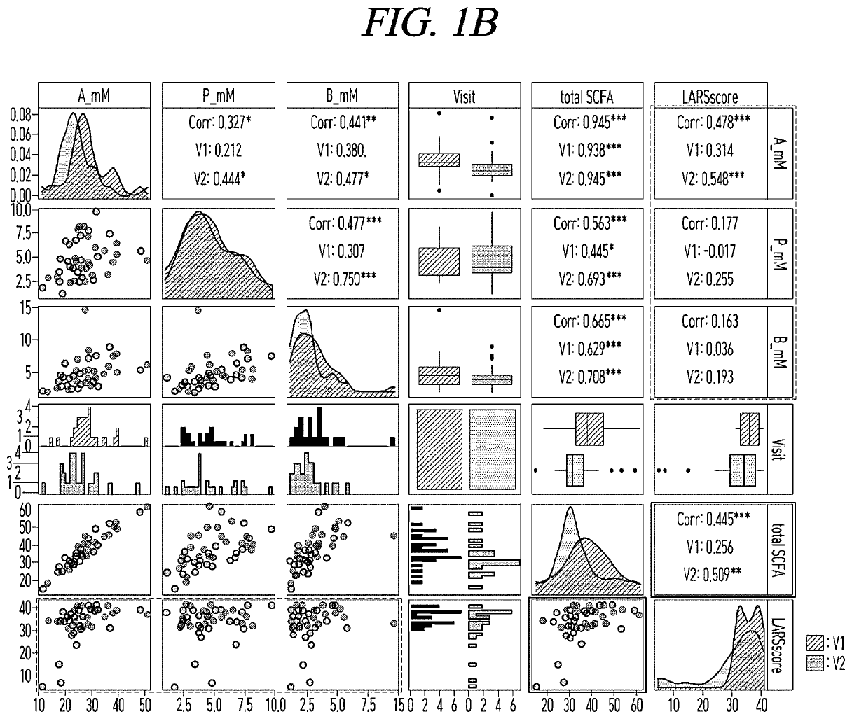

Fecal samples (from a total of 26 study subjects before and after intake) were weighed to 0.2 g using an electronic balance, added with 1 mL of distilled water with sufficient stirring, and homogenized. After centrifugation at 13,000 rpm and 4° C. for 10 minutes, 150 μL of supernatant was transferred into a gas chromatography (GC) vial and 150 μL of a GC buffer solution was dispensed, followed by flame ionization detector (GC-FID) analysis (FIG. 1A). [00104]100 μL of a GC buffer solution [composition: 10 mL of distilled water, 8.82 g of (NH₄)₂SO₄, 2.38 g of monosodium phosphate, 50 μL of phosphoric acid, and 0.5 μL of 2-ethyl butyric acid] for metabolite analysis, 50 μL of distilled water, and 50 μL of supernatant were dispensed to a GC vial. Then, six (6) types of SCFAs (acetate, propionate, butyrate, isobutyrate, valerate, and isovalerate), which are microbial metabolites, were analyzed using GC-FID.

The total SCFA level was calculated by summing the quantitative values of acetate, propionate, and butyrate, and the Wilcoxon signed-rank test was conducted to compare SCFA levels before and after intake in each of the 26 study subjects.

The Wilcoxon signed-rank test was conducted on the 26 study subjects to evaluate the significance of differences in score before and after intake. The result showed that the composition of the present disclosure significantly reduced the levels of total SCFA and acetate among the six types of SCFAs, which leads to improved bowel movement and alleviation of abnormal bowel habits, such as diarrhea and frequent bowel movements.

### (2) Correlation Between Fecal SCFA and LARS Score

The correlations between SCFA levels and LARS scores before and after intake by the 26 study subjects was analyzed. The scatter plots visualize the correlations between fecal SCFA levels (total SCFA, AmM, PmM, and BmM) and LARS scores before and after intake of the composition according to the present disclosure by the study subjects, and the correlations were calculated and expressed using the Pearson correlation coefficients (FIG. 1B). In FIG. 1B, shaded or black-filled graphs represent before intake V1, while dotted graphs represent after intake V2.

The total SCFA showed a significant positive correlation with LARS scores (solid box, Pearson R=0.445), and among three major SCFAs, acetate showed a significant positive correlation (Pearson correlation R=0.478) with a LARS score (correlation and scatter plot between each SCFA level and LARS score; dotted box).

### Example 2: Comparison of SCFA Levels of Normal Group and LARS Patient Group without Intake of Strain

Fecal samples (from 26 study subjects in each group) of a normal group and a LARS patient group were weighed to 0.2 g using an electronic balance, added with 1 mL of distilled water with sufficient stirring, and homogenized. Then, after centrifugation at 13,000 rpm and 4° C. for 10 minutes, 150 μL of supernatant was transferred into a GC vial and 150 μL of a GC buffer solution was dispensed, followed by GC-FID analysis.

100 μL of a GC buffer solution [composition: 10 mL of distilled water, 8.82 g of (NH₄)₂SO₄, 2.38 g of monosodium phosphate, 50 μL of phosphoric acid, and 0.5 μL of 2-ethyl butyric acid] for metabolite analysis, 50 μL of distilled water, and 50 μL of supernatant were dispensed to a GC vial. Then, six (6) types of SCFAs (acetate, propionate, butyrate, isobutyrate, valerate, and isovalerate), which are microbial metabolites, were analyzed using GC-FID.

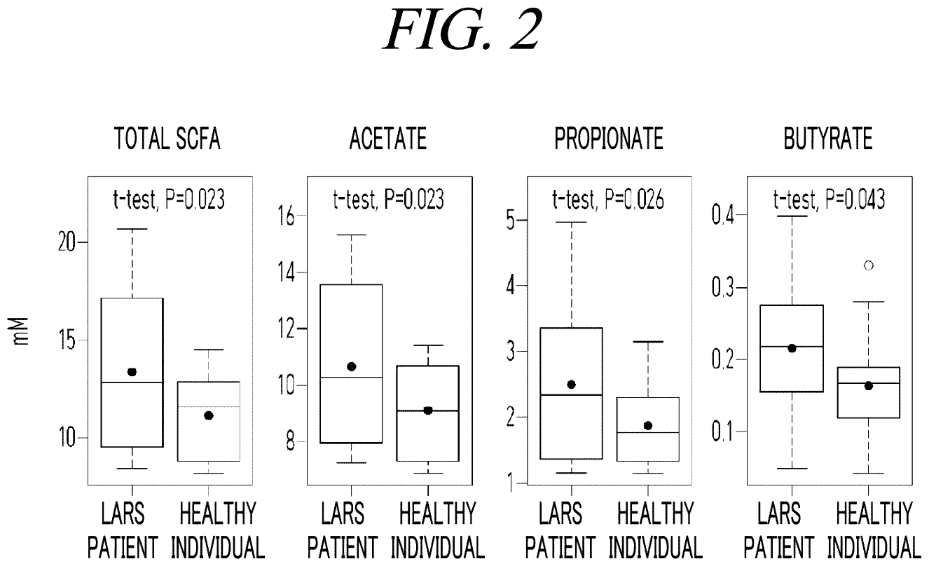

The total SCFA level was calculated by summing the quantitative values of acetate, propionate, and butyrate, and the Independent Samples t-Test was conducted to compare the means of the total SCFA, acetate, and butyrate levels in the feces of the normal group and the LARS patient group (black dots in the graph of FIG. 2).

The result showed that overall fecal SCFA levels were significantly higher in the LARS patient group than in the healthy group (FIG. 2).

### Example 3: Comparison of Fecal SCFA Levels of Normal Group and LARS Patient Group with Intake of Strain

### (1) Culture of Strain HEM1036 Using Personalized Pharmaceutical Meta-Analysis Screening (PMAS) Technique

To check production amounts of SCFAs by Lactobacillus fermentum HEM1036 of the present disclosure, in vitro screening culture with fecal samples from the LARS patient group was conducted using the PMAS technique (see Korean Patent. Nos. 10-2227382 and 10-2124474) developed by HEM Pharma Inc. (i.e., the present applicant).

A gut environment-like medium contains NaCl, NaHCO₃, KCl, hemin, mucin, L-cysteine, and resazurin (hereinafter, referred to as “PMAS medium”).

After the PMAS medium containing the above-described composition was prepared, it was anaerobically substituted for 24 hours in a Whitley A95 anaerobic workstation to create an anaerobic environment.

The feces of the normal group or the LARS patient group were homogenized in the PMAS medium within the Whitley A95 anaerobic workstation, and a filter was used to filter out residues of the feces.

In a 96-well plate, the homogenized samples were dispensed into wells for the control group NC (Negative Control), and the homogenized samples and Lactobacillus fermentum HEM1036 were dispensed into wells for the test group.

Then, the 96-well plate for the test group and the control group was cultured with stirring using a stirrer in the Whitley A95 anaerobic workstation for 24 hours.

### (2) Comparison of Production Amounts of SCFAs

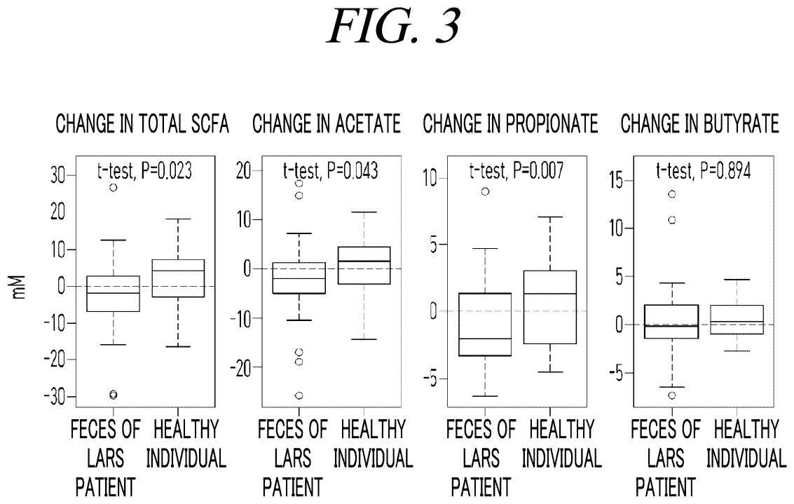

After PMAS screening culture performed as described above, centrifugation was performed at 3,800 rpm and room temperature for 10 minutes to transfer supernatant of each well of the 96-well plate to a 96-well cell culture plate. Thereafter, 100 μL of a GC buffer solution [GC buffer solution composition: 10 mL of distilled water, 38.82 g of (NH₄)₂SO₄, 2.38 g of monosodium phosphate, 50 μL of phosphoric acid, and 0.5 μL of 2-ethyl butyric acid] for metabolite analysis, 50 μL of distilled water, and 50 μL of supernatant were dispensed to a GC vial. Then, six (6) types of SCFAs (acetate, propionate, butyrate, isobutyrate, valerate, and isovalerate), which are microbial metabolites, were analyzed using GC-FID (FIG. 3).

The changes in SCFA ratio after treatment with the strain were confirmed by comparing the result thereof with the result of Example 2 (FIG. 2 and FIG. 3). Further, non-cultured fecal samples were analyzed using GC-FID to examine the changes in SCFAs with or without culturing.

The non-cultured fecal samples were weighed to 0.2 g using an electronic balance, added with 1 mL of distilled water with sufficient stirring, and homogenized. Then, after centrifugation at 13,000 rpm and 4° C. for 10 minutes, 150 μL of supernatant was transferred into a GC vial and 150 μL of a GC buffer solution was dispensed, followed by GC-FID analysis.

The changes in SCFAs were calculated by comparing the differences between wells treated with HEM1036 and the control wells (PMAS culture wells without strain treatment). The means of the changes in SCFAs in the fecal samples from the LARS patient group and the changes in SCFAs in the fecal samples from the healthy group were compared by the Independent Samples t-Test, and P-values were indicated. If the values are below the dashed line (y=0), it indicated that the SCFA levels decreased in the HEM1036-treated PMAS wells compared to the control group.

The analysis confirmed a significant reduction in levels of total SCFA and acetate among the six types of SCFAs.

### Example 4: Comparison of LARS Scores of LARS Patient Group Before and After Intake of Strain

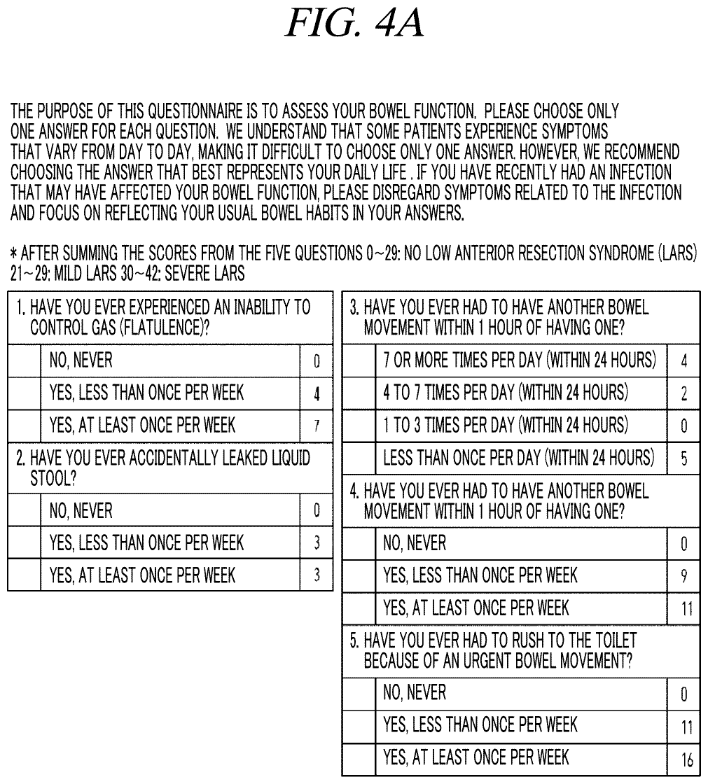

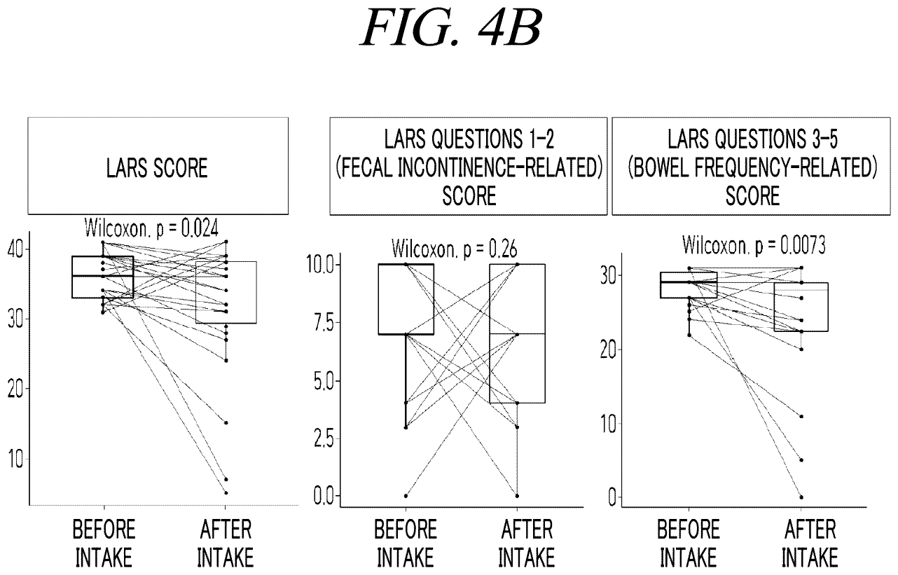

LARS questionnaire response scores of the LARS patient group were compared before and after intake of HEM1036. The Wilcoxon signed-rank test was conducted to evaluate the significance of differences in score of the 26 study subjects before and after intake (FIG. 4A). As a result, it was confirmed that the intake of HEM1036 significantly improved the LARS score.

According to the study by Kim et al. (2021), the LARS questionnaire items can be classified into questions related to fecal incontinence symptoms (Q1-Q2) and questions related to frequent bowel movement symptoms (Q3-Q5). In particular, it was observed that the intake of HEM1036 significantly improved frequent bowel movement symptoms (FIG. 4B).

### Example 5: Comparison of Questionnaire Response Scores of Cancer Patient Group Before and After Intake of Strain

Quality of life questionnaire response scores of a colorectal cancer patient group were compared before and after intake of the composition according to the present disclosure. The Wilcoxon signed-rank test was conducted to evaluate the significance of differences in score of the 26 study subjects before and after intake. The result of comparing all indicators related to quality of life (Global health status/QoL) and functional scales as well as symptom scale indicators except for nausea and vomiting, dyspnoea, and financial difficulties before and after intake were shown in the drawing.

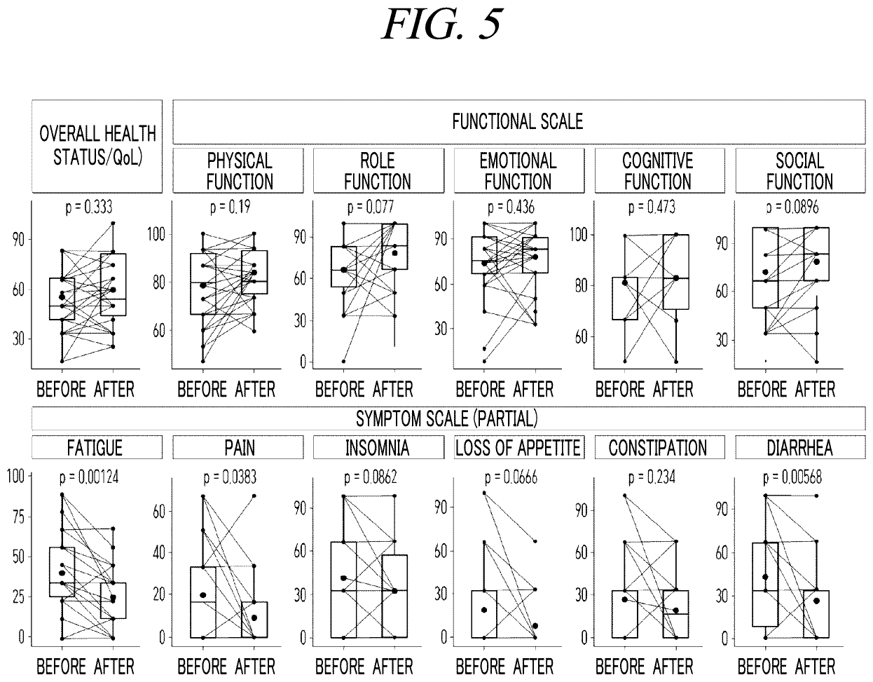

As a result, it was confirmed that the intake of HEM1036 generally improved the quality of life and functional scales and also improved the symptom scales (FIG. 5). In particular, it was confirmed that the intake of HEM1036 significantly improved diarrhea among the symptom scales.

### Example 6: Confirmation of Changes in Gut Microbiota Richness in Study Subject Group

### (1) Analysis of Gut Microbiota Richness

A gut microbiome analysis of fecal samples (from a total of 26 study subjects before and after intake) was conducted by extracting all genomes from the fecal samples and performing genome-based 16S rRNA V3-V4 region Next generation sequencing (NGS) analysis using the Illumina MiSeq System with bacteria-specific primers. The sequencing results were preprocessed in Qiime2 using DADA2 denoising.

A statistical analysis was conducted using the phyloseq package in R (ver. 4.1.1) to calculate alpha diversity from the sequencing result of each fecal sample. The Wilcoxon signed-rank test was conducted to compare the alpha diversity of fecal microbiome before and after intake in each of the 26 study subjects.

### (2) Analysis Result

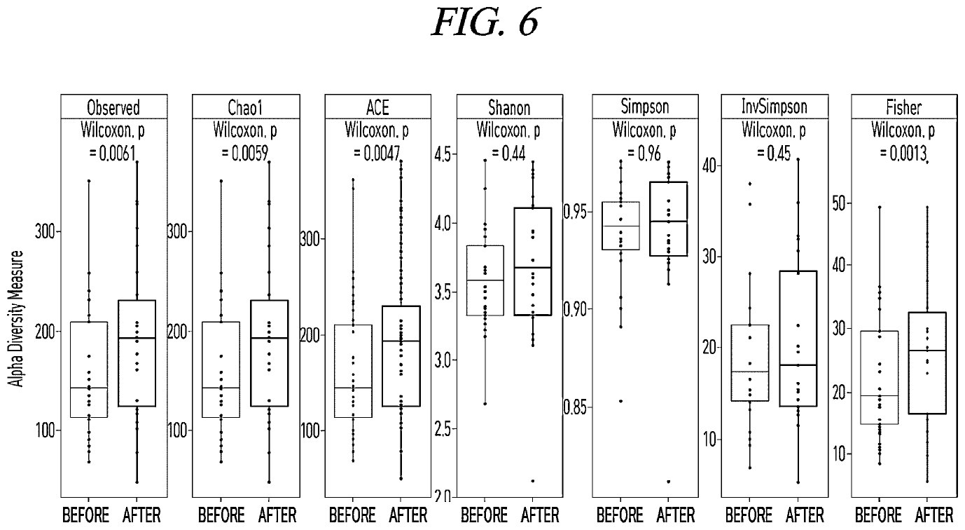

The alpha diversity of the gut microbiota (microbiome) in the fecal samples before and after intake of HEM1036 was compared, and the Wilcoxon signed-rank test was conducted to evaluate the significance of differences in score of the 26 study subjects before and after intake. The richness indices of gut microbiota, such as Observed, Chaol, A C E, and Fisher, showed a significant increase after intake of HEM1036. Also, indices, such as Shannon and Simpson, used to assess both richness and homogeneity exhibited an increasing trend, although not statistically significant, after intake (FIG. 6).

### Example 7: Confirmation of Amelioration of Diarrhea and Weight Loss of Mouse Model by Administration of HEM1036

To verify amelioration of chemotherapy-induced diarrhea by Lactobacillus fermentum HEM1036 of the present disclosure, a test was conducted as described below.

### (1) Animal Breeding

Six-week-old male BALB/c mice were divided into three groups for test: a control group (Ctrl), an anti-cancer agent-induced diarrhea group (5-FU), and an anti-cancer agent-induced diarrhea group treated with HEM1036 (1036).

Four-week-old male BALB/c mice were purchased and acclimated to the vivarium for two weeks. Then, L. fermentum HEM1036 was administered to them for a total of three weeks. Thereafter, 5-FU was intraperitoneally injected into the mice daily for five days and then washed out for two days before sacrificing the animals.

TABLE 1

| | | | CID treatment | | |
|---|---|---|---|---|---|
| | | Number of | (Intraperitoneal | Administration of | |
| No. | Group name | animals | injection) | substance (Oral) | Remarks |
| 1 | Ctrl | 7 | 1X PBS | 1X PBS 200 | Normal control |
| | | | | μl/day | group |
| 2 | 5-FU | 8 | 5FU 30 mpk | 1X PBS 200 | Anti-cancer |
| | | | | μl/day | agent-induced |
| | | | | | diarrhea group |
| 3 | 1036 | 8 | 5FU 30 mpk | HEM1036 1 × 10⁹ | HEM1036- |
| | | | | CFU/200 μl/day | administered |
| | | | | | group |

### (2) Tissue Analysis

At the time of animal sacrifice, blood, colon, cecum, small intestine, and feces were extracted from the mice, and the expression levels and protein levels of diarrhea- and inflammation-related markers were checked by quantitative real-time polymerase chain reaction (qRT-PCR), general histological staining, and immunohistochemistry. Statistical significance was analyzed by one-way ANOVA (Dunnett's comparison).

(3) Confirmation of Effect of Lactobacillus fermentum HEM1036 on Amelioration of Anti-Cancer Agent-Induced Diarrhea and Weight Loss

To evaluate the degree of amelioration of weight loss and diarrhea, which are side effects of administration of anti-cancer agents, the extents of weight loss and diarrhea scores induced by 5-FU in mice orally administered with PBS or HEM1036 for three weeks were compared.

The body weights of the mice were measured before administration of the anti-cancer agent and set as a reference weight. Thereafter, 5-FU was intraperitoneally injected once daily at a dose of 30 mpk, and the extents of weight loss as well as the severity of diarrhea in feces were monitored and scored. After sacrificing the animals, the length of the colon was measured and fecal clumps in the colon were examined to validate the appropriateness of the diarrhea scores. Lastly, H&E staining was performed on the colon tissue to compare the tissue morphology and inflammation status.

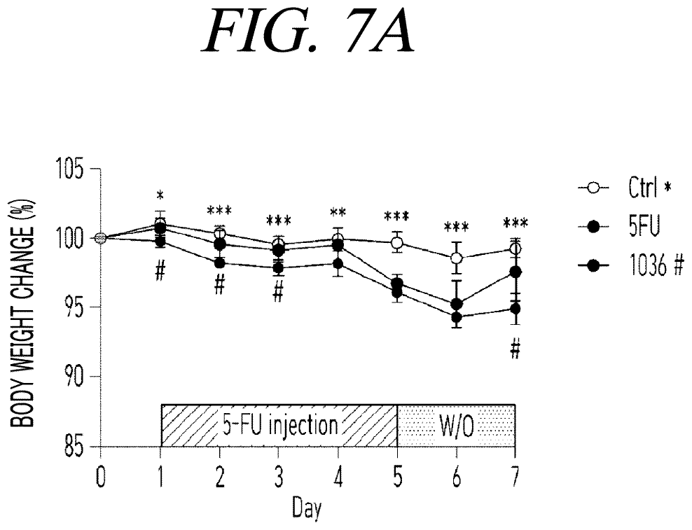

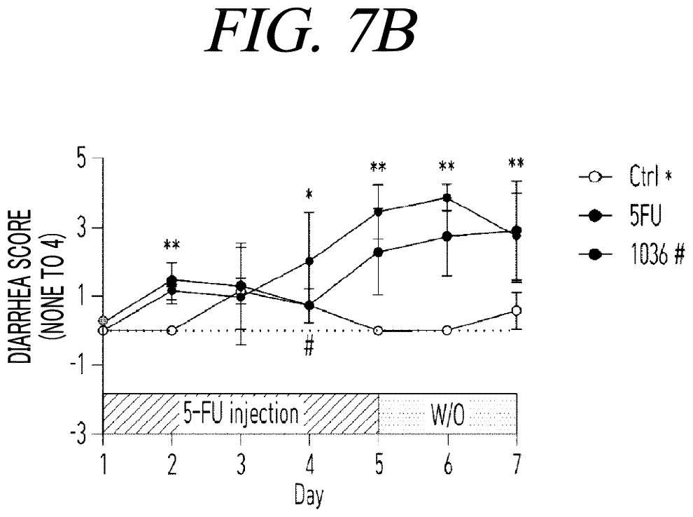

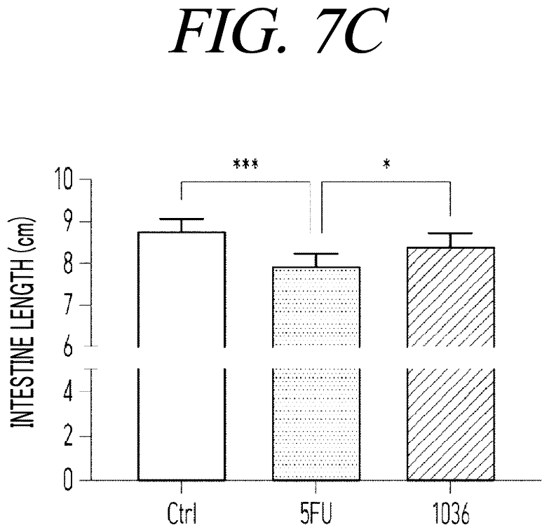

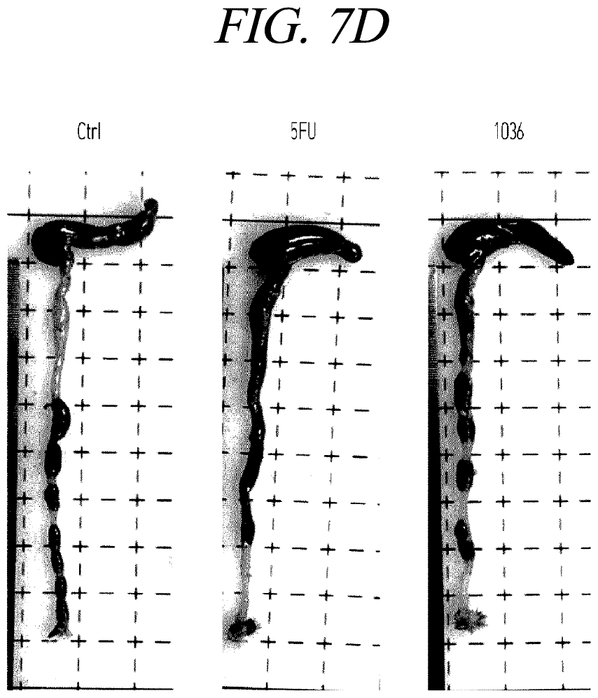

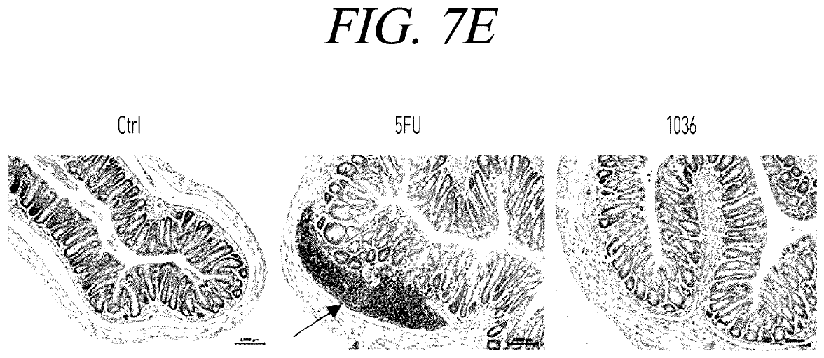

As a result, it was confirmed that the L. fermentum HEM1036-administered group showed a significant inhibition of weight loss compared to the disease-induced control group (FIG. 7A), the diarrhea score and colon length were significantly improved (FIG. 7B, FIG. 7C and FIG. 7D), and the degree of inflammation of the tissue was reduced (FIG. 7E).

Based on the above result, it can be seen that L. fermentum HEM1036 of the present disclosure is effective in ameliorating weight loss and diarrhea, which are side effects of administration of anti-cancer agents.

### Example 8. Confirmation of Factors that Affect Amelioration of LARS, Diarrhea, or Weight Loss

Changes in factors, such as intestinal permeability, small intestinal villi length, and electrolyte balance indicator, which can affect amelioration of LARS, diarrhea, or weight loss were checked.

### (1) Confirmation of Ability to Improve Intestinal Permeability and Inflammatory Marker

Mouse models and analysis information were identical to those described in the above paragraphs (1) and (2) of Example 7.

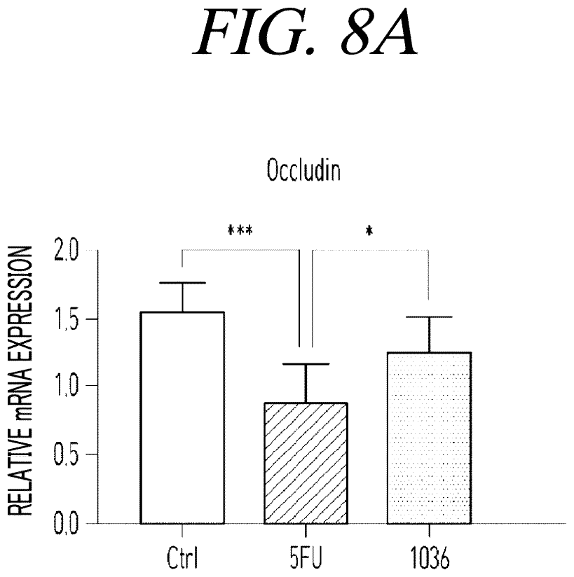

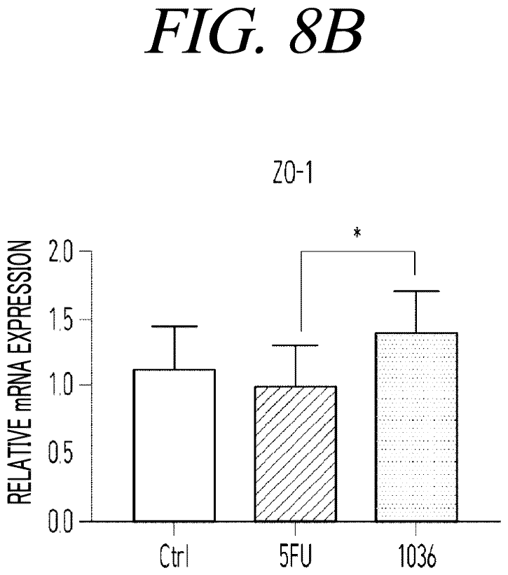

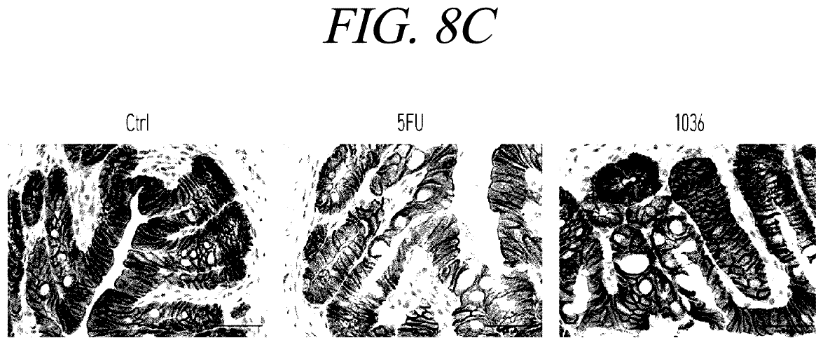

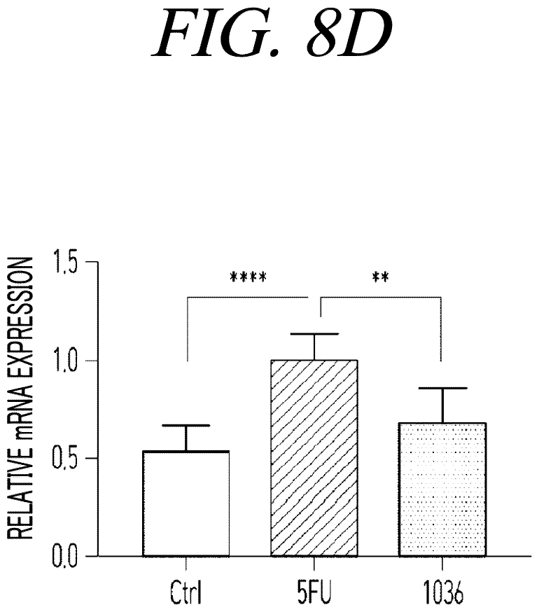

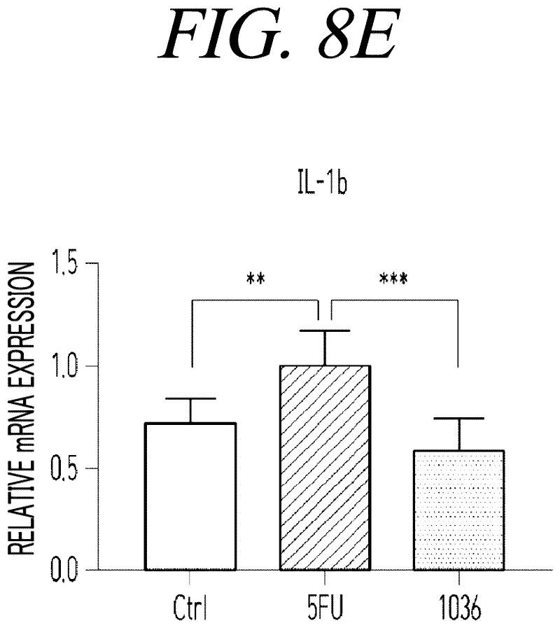

To determine whether an increase in intestinal permeability and inflammation observed in the colon after administration of an anti-cancer agent can be improved by administration of the strain L. fermentum HEM1036 of the present disclosure, changes in relevant markers were examined in the colon extracted from the sacrificed animals. When the expression levels of tight junction proteins occludin and ZO-1, which are closely related to intestinal permeability, were analyzed by qRT-PCR, the result showed a significant improvement by administration of L. fermentum HEM1036 (FIG. 8A and FIG. 8B). Also, an increase in protein level was confirmed by immunohistochemistry for occludin in the colon tissue (FIG. 8C). Further, when the expression levels of TNF-α and IL-1β, which are inflammatory cytokines, were checked by qRT-PCR, the result showed a significant decrease compared to the disease-induced control group (FIG. 8D and FIG. 8E).

### (2) Confirmation of Improvement of Small Intestinal Villi Length

Mouse models and analysis information were identical to those described in the above paragraphs (1) and (2) of Example 7.

To determine whether a decrease in small intestinal villi length observed in the diarrhea models can be improved by administration of the strain L. fermentum HEM1036 of the present disclosure, H&E staining was performed on the jejunum of the small intestine extracted from the sacrificed animals. The lengths of the villi and crypts were measured and compared using ImageJ Software.

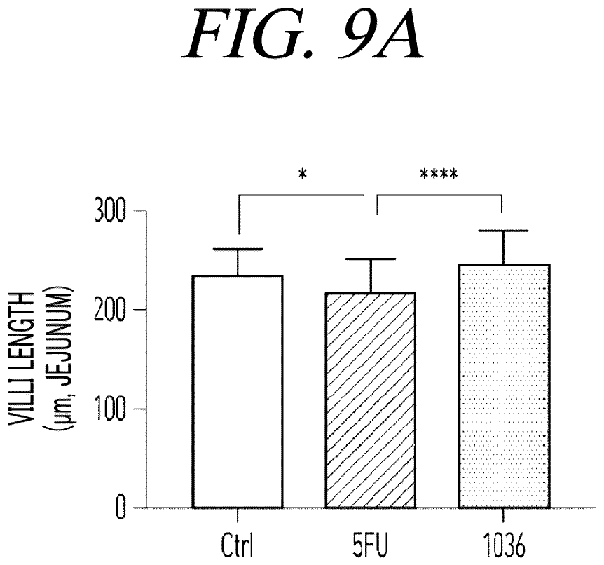

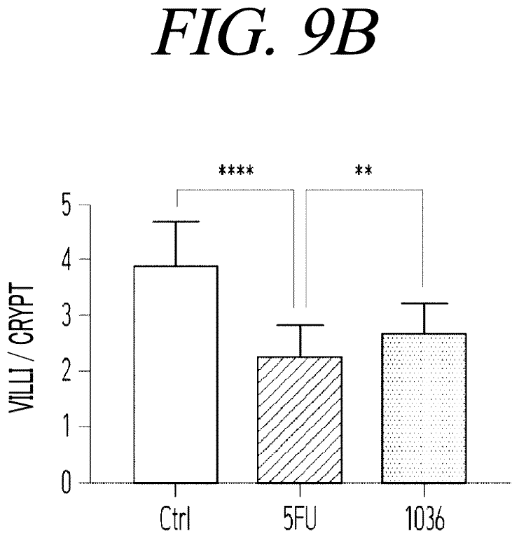

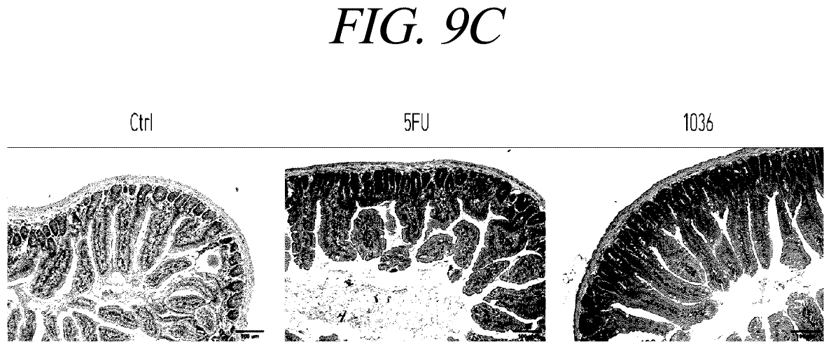

As a result, it was confirmed that the L. fermentum HEM1036-administered group showed a significant improvement of villi length and villi/crypt ratio compared to the disease-induced control group (FIG. 9A, FIG. 9B and FIG. 9C).

### (3) Confirmation of Change in Electrolyte Balance Indicator

Mouse models and analysis information were identical to those described in the above paragraphs (1) and (2) of Example 7.

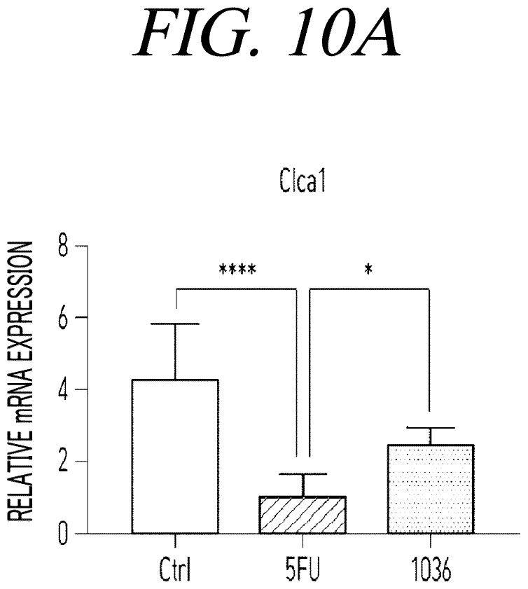

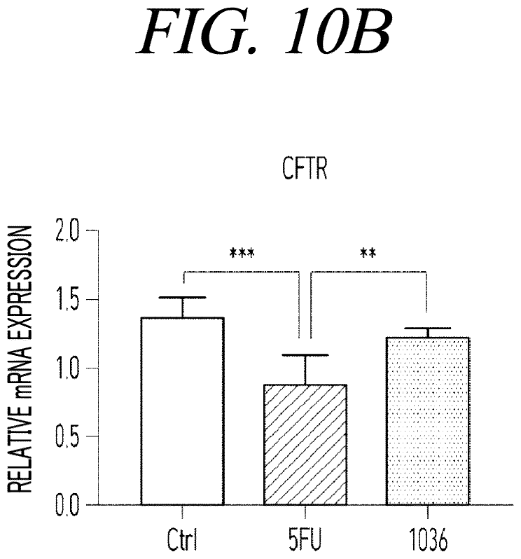

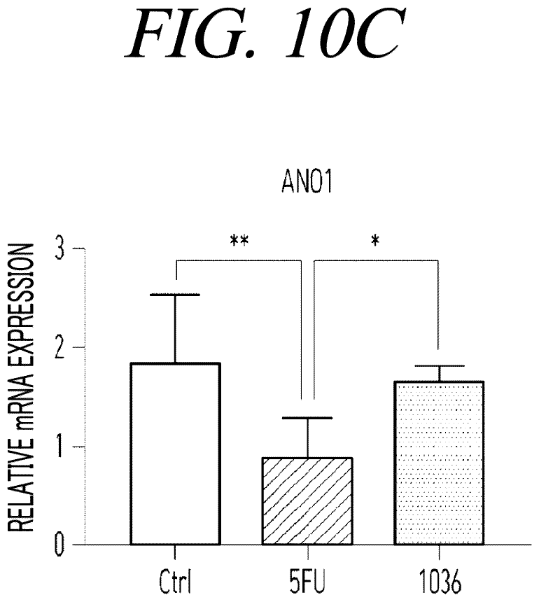

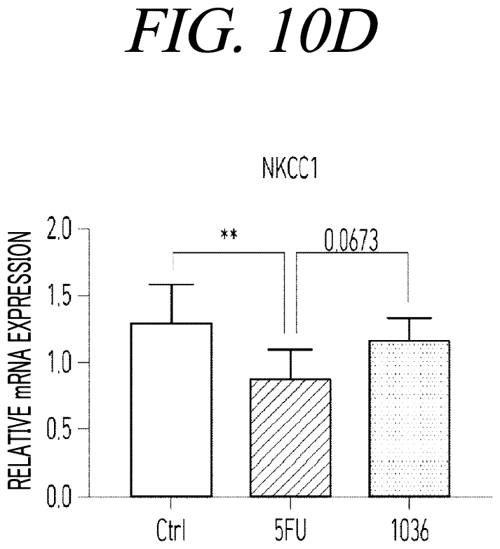

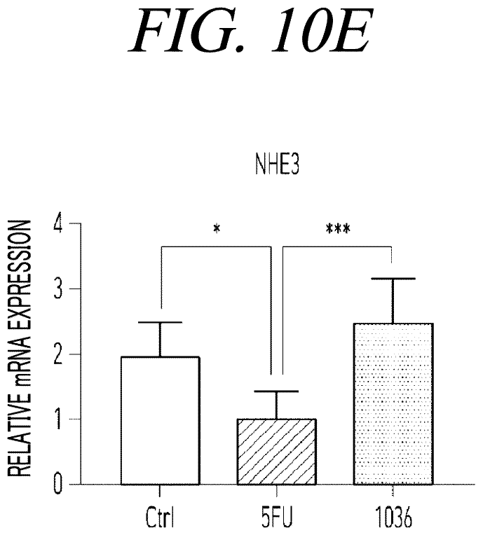

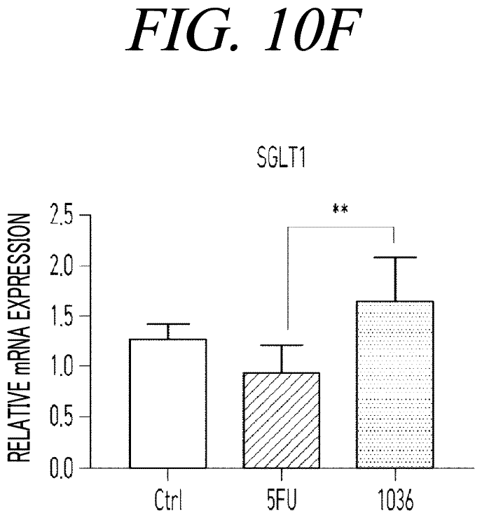

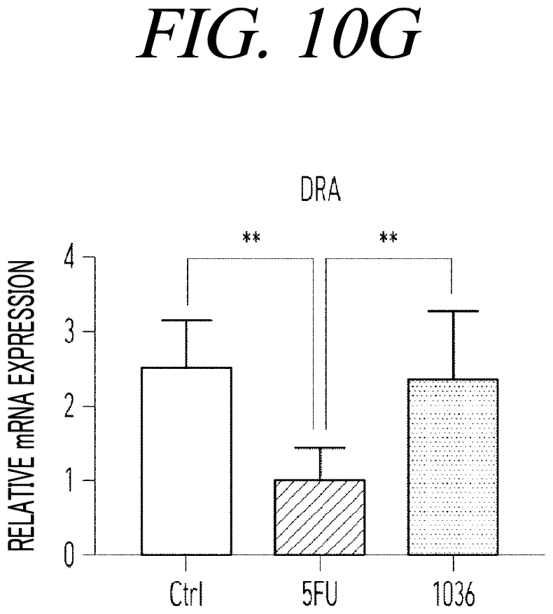

To determine whether an imbalance in electrolyte balance indicator observed in the diarrhea models can be improved by administration of the strain L. fermentum HEM1036 of the present disclosure, changes in electrolyte transport-related proteins in the colon extracted from anti-cancer agent-administered models were checked by qRT-PCR. As a result, it was confirmed that the expression levels of electrolyte transport proteins Clca1, CFTR, ANO1, NKCC1, NHE3, SGLT1, and DRA were decreased in the disease-induced control group, but were significantly increased by administration of L. fermentum HEM1036 (FIG. 10A to FIG. 10G).

### Example 9. Confirmation of Effect of HEM1036 on Inhibition of Weight Loss of Colorectal Cancer Mouse Model

### (1) Animal Breeding

Six-week-old male BALB/c mice were divided into the following groups for test: a normal control group (NC), a cancer-induced control group (DC), an anti-cancer agent-administered control group (5-fluorouracil (5-FU)), HEM1036 low-dose treatment group, and HEM1036 high-dose treatment group

TABLE 2

| | | | Anti-cancer | | |
|---|---|---|---|---|---|
| | | | agent treatment | | |
| | | Number of | (Intraperitoneal | Administration of | |
| No. | Group name | animals | injection) | substance (Oral) | Remarks |
| 1 | NC | 8 | 1X PBS | 1X PBS 200 | Normal control |
| | | | | μl/day | group |
| 2 | DC | 8 | 1X PBS | 1X PBS 200 | Cancer-induced |
| | | | | μl/day | control group |
| 3 | 5FU | 8 | 5FU 30 mpk/ | 1X PBS 200 | Anti-cancer |
| | | | every other day | μl/day | agent- |
| | | | | | administered |
| | | | | | control group |
| 4 | Low | 8 | 5FU 30 mpk/ | HEM1036 1 × 10⁸ | HEM1036 low- |
| | | | every other day | CFU/200 μl/day | dose treatment |
| | | | | | group |
| 5 | High | 8 | 5FU 30 mpk/ | HEM1036 1 × 10⁹ | HEM1036 high- |
| | | | every other day | CFU/200 μl/day | dose treatment |
| | | | | | group |

CT26 mouse colorectal cancer cells (murine colorectal carcinoma) were percutaneously injected once at a dose of 5×10⁵ cells/mouse, and HEM1036 was orally administered every day from the next day. Also, the body weights and tumor sizes were measured while 5-FU was intraperitoneally injected at two-day intervals, throughout the experiment. The animals were bred for a total of three weeks and then sacrificed. The body weights and tumor weights were measured at the time of animal sacrifice.

(2) Confirmation of Effect of Lactobacillus fermentum HEM1036 on Inhibition of Weight Loss of Cancer Chemotherapy Model

The mouse models had cancer induced by injecting CT26, which is a colorectal cancer cell line derived from the BALB/c mice and were subjected to cancer chemotherapy by treatment with 5-FU, which is a chemotherapy agent. To check whether weight loss which is a side effect caused by the anti-cancer agent in the mouse models can be inhibited by administration of L. fermentum HEM 1036 of the present disclosure, L. fermentum HEM 1036 was administered at a low dose (1×10⁸ CFU/mouse/day) and a high dose (1×10⁹ CFU/mouse/day) and changes in body weight and tumor size were measured.

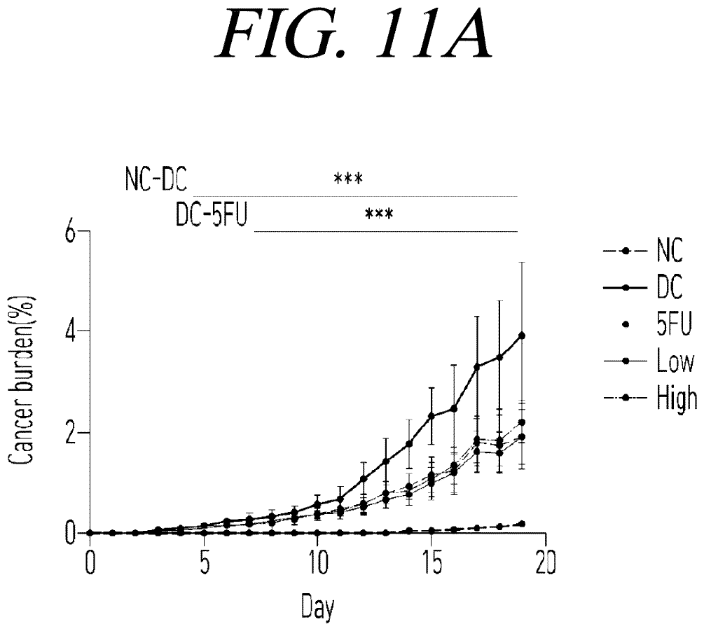

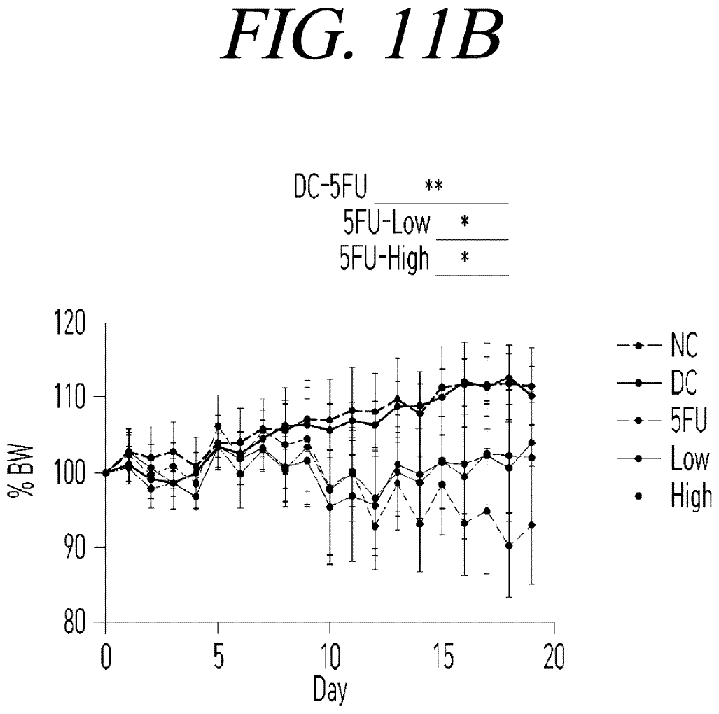

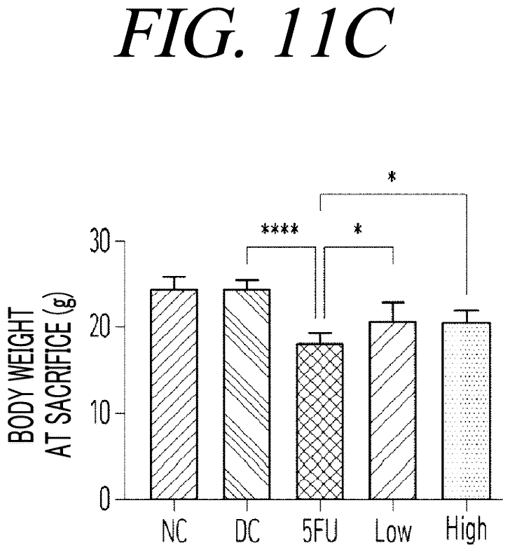

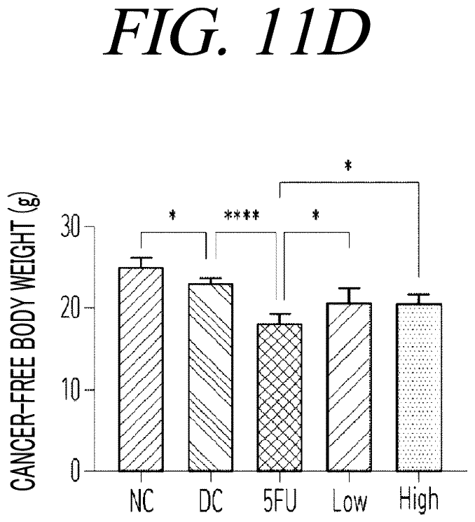

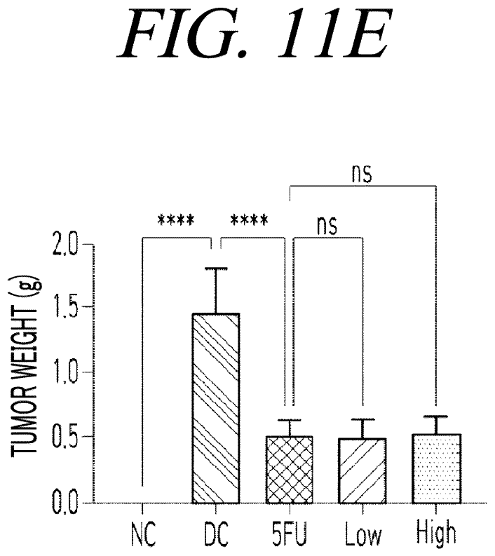

The result showed that administration of L. fermentum HEM1036 did not cause any changes in tumor size or weight, but significantly inhibited weight loss and a decrease in cancer-free body weight compared to the anti-cancer agent-administered control group (FIG. 11A to FIG. 11E).

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description. It shall be understood that all modifications and embodiments conceived from the meaning and scope of the claims and their equivalents are included in the scope of the present disclosure.

We claim:

1. A composition for ameliorating side effects of cancer treatment, comprising a strain Lactobacillus fermentum HEM1036 deposited with the Korean Collection for Type Cultures under Accession No. KCTC13978BP as an active ingredient,
wherein the side effects of cancer treatment include low anterior resection syndrome (LARS), diarrhea, or weight loss.

2. The composition of claim 1,
wherein the composition decreases the levels of total short-chain fatty acids (total SCFA) and acetate.

3. The composition of claim 1,
wherein the composition decreases the expression level of TNFα or IL-1β.

4. The composition of claim 1,
wherein the composition increases the expression of one or more selected from the protein group consisting of Clca1, CFTR, ANO1, NKCC1, NHE3, SGLT1, and DRA.

5. The composition of claim 1,
wherein the composition comprises a strain Lactobacillus fermentum HEM1036, live cells, heat-killed cells, culture fluid, fragments, or extracts thereof.

6. A pharmaceutical composition for ameliorating side effects of cancer treatment, comprising a strain Lactobacillus fermentum HEM1036 deposited with the Korean Collection for Type Cultures under Accession No. KCTC13978BP as an active ingredient,
wherein the side effects of cancer treatment include LARS, diarrhea, or weight loss.

7. A method for ameliorating side effects of cancer treatment, comprising:
administering the composition of claim 1.
